(19) United States
(12) Patent Application Publication
(10) Pub. No.: US 2025/0345544 A1
(43) Pub. Date: Nov. 13, 2025

(54) MEDICAL INSTRUMENT FOR INTUBATION

(71) Applicant: Albert-Ludwigs-Universität Freiburg, Freiburg (DE)

(72) Inventors: Ralf PETZOLD, Freiburg (DE); Martin SCHICK, Freiburg (DE)

(21) Appl. No.: 18/862,226

(22) PCT Filed: Apr. 25, 2023
(86) PCT No.: PCT/EP2023/060745

§ 371 Date: Nov. 1, 2024

(30) Foreign Application Priority Data
May 4, 2022 (EP) 22171524.6

(51) Int. Cl. A61M 16/04

(52) U.S. Cl. CPC A61M 16/0488; A61M 2205/0216; A61M 2205/586

(57) ABSTRACT

A medical instrument used for intubating a patient with an endotracheal tube, ETT for short, enclosing a hollow channel, and also with a guiding rod, which is arranged along the hollow channel, which is plastically deformable at least in an axial portion and has a distal rod end and a proximal rod end, attached to the distal rod end of which guiding rod is a bidirectionally deformable guiding element, which has a distal element end and a proximal element end. The proximal element end of the guiding rod element is joined to the distal rod end and the guiding element is connected to a control. The invention has an operating unit attached to the proximal rod end of the guiding rod and an actuator, connected to the control, and has a push-pull mechanism, in operative releasable connection with the ETT by which the ETT can be moved along the guiding rod, for intubing the ETT together with the guiding rod arranged in the hollow channel of the ETT along with the bidirectionally deformable guiding element attached thereto. The distal end of the ETT is dome shaped.

### CROSS-REFERENCE TO RELATED APPLICATIONS

Reference is made to PCT/EP2023/060745 filed Apr. 25, 2023 and European Patent Application No. 22 171 524.6 filed May 4, 2022, which are incorporated herein by reference in their entirety.

### BACKGROUND OF THE INVENTION

### Field of the Invention

The invention relates to a medical instrument for the intubation of a patient with an endotracheal tube, ETT for short, enclosing a hollow channel, and a guiding rod arranged along the hollow channel and which is plastically deformable at least in an axial portion, with a distal and proximal rod end, to the distal rod end of which a bidirectionally deformable guiding element is attached, with a distal and a proximal element end with the proximal element end being joined to the distal rod end, and which is in operative connection with a control means.

To secure the airway for general anaesthesia, or in emergency, or intensive care medicine, intubation anaesthesia, or ITN for short, is, and remains, a standard procedure for which various aids are required in order to position a breathing tube or endotracheal tube, ETT for short, into the patient's trachea. The ETT must be advanced through the open mouth, throat and then between the vocal cords into the windpipe (trachea). During this procedure, the view of the vocal cords, which is usually obtained with the aid of a laryngoscope, is rudimentary. During this phase, the patient cannot be ventilated, so a quick and smooth procedure is very important, if not vital, for the patient. The patient can only be ventilated safely by way of the correctly positioned ETT.

If intubation is not possible at the first attempt, or is only possible with difficulty, this is referred to as difficult airway management, or difficult intubation. In adults, this occurs in 3-10% of all intubations performed. See Ruetzler K, Smereka J, Abelairas-Gomez C, Frass M, Dabrowski M, Bialka S, et al, Comparison of the new flexible tip bougie catheter and standard bougie stylet for tracheal intubation by anaesthesiologists in different difficult airway scenarios: a randomised crossover trial. BMC Anaesthesia. 2020; 20(1):90.

In Germany alone, around 24 million operations are performed each year, of which around 70% are supported and accompanied by general anaesthesia, meaning that around 0.5 to 1.6 million patients are affected by this problem in Germany each year.

To facilitate and support the insertion and positioning of the ETT in the pharynx, and protruding through the vocal cords into the upper tracheal area, a so-called guiding rod (in English also called a bougie or stylet) is either positioned inside the ETT to shape it, or inserted into the trachea in advance instead of the ETT, in order to advance the ETT into the trachea immediately afterwards, via the already inserted guiding rod. The patient cannot be ventilated during the entire intubation process. To ensure the patient's oxygen supply, it may be necessary to interrupt the intubation process from time to time, and ventilate the patient with a mask, for example.

The use of a guiding rod is particularly advantageous in cases where a good view of the vocal cords is possible, for the most part using a laryngoscope, but despite a suitable patient head position, the oral, pharyngeal and tracheal airway axes of the patient are unfavourably oriented with respect to each other, such that the soft preformed tube achieved in production cannot be positioned in a suitable manner.

In these cases, the individual guiding rod is initially bent in an empirical manner to match the anatomical conditions, which often means that a number of attempts at intubation have to be made with the initially bent guiding rod inserted in the ETT. The bend has to be adjusted after each failed intubation attempt until the correct angle is found, and the ETT can be positioned in the trachea. This procedure can sometimes take several minutes.

Another application of the guiding rod is found in the so-called Rapid Sequence Induction (RSI). This process involves a rapid intubation of patients after the introduction of anaesthesia, who are, for example, at high risk of aspiration, that is from the vomiting of stomach contents, and the subsequent penetration of these components into the lungs. To enable a rapid and successful intubation in this case, a guiding rod is positioned in the ETT in a standard procedure during the first attempt, and is pre-shaped based on the presumed anatomy. Here too, as mentioned above, the intubation procedure may have to be aborted and the guiding rod and ETT may have to be pre-shaped once again. Here there is not only a risk of oxygen deprivation due to the lack of ventilation, but also a potentially fatal aspiration of stomach contents.

After a successful positioning of the ETT, including the guiding rod, in most cases shortly upstream or downstream of the vocal cord plane, the guiding rod is either gradually withdrawn, or completely removed, by a second person. During this procedure, the correctly positioned ETT may slip as a result of this manipulation due to the high frictional forces between the ETT and the guiding rod, making it no longer possible to insert it into the trachea. Rarely is it possible to reinsert the guiding rod into the ETT in situ, meaning that the procedure has to be aborted, and the process restarted from the beginning, with all the risks as described above.

The guiding rod is an item in the minimum equipment of an anaesthesia workstation, as required by the German Society of Anaesthesiology and Intensive Care Medicine (S1 Guideline on Airway Management, see www.awmf.org).

In addition to the plastic deformability of the guiding rod, the guiding rod to be initially bent manually to facilitate its insertion and positioning through the pharynx and vocal cords into the upper tracheal region. A very few modern guiding rods also have a distal rod section that can be deformed in a controlled manner, the spatial curvature of which can be altered to facilitate the insertion of the guiding rod in a manner that is gentle for the patient.

In the publication EP 3 065 803 B1, a guiding rod or bougie is disclosed, which has a tubular, plastically deformable main shaft, at the distal end of which is attached a movable tip which can be deformed bidirectionally in a deformation plane by means of a control unit mounted for longitudinal movement within the main shaft. For this purpose, the tubular main shaft has a recess extending in the longitudinal direction, through which projections connected to the control unit protrude, which are manually accessible to the operator.

EP 3 528 878 B1 describes an articulated mandrel for use with an endotracheal tube, wherein the mandrel has a flexible distal tip section which is preloaded in a first direction and can be deformed in a second direction, opposite to the first direction, by means of a control wire connecting the distal tip section to a proximal mandrel section, by performing an appropriate tensile action.

A guiding rod very similar to the above mandrel can be found in WO 2007/138569 A2 which has a rigid rod section and an adjoining flexible rod section, at the distal end to which is attached a pivoting element that is mounted such that it can pivot about an axis, for the one-sided deflection of which is provided a wire cable transferring tensile forces, which is connected on the one hand to the pivoting element, and on the other hand to a lever mechanism which is attached to the proximal section of the guiding rod. For purposes of intubation, the guiding rod, together with the endotracheal tube that surrounds it, is inserted orally into the patient.

WO 2017/079434 A1 discloses a guiding rod with an elastically deformable distal guiding rod tip, to which at least one wire, preferably a number of wires, are attached, transmitting tensile forces which are connected to a handle body attached to the proximal end of the guiding rod as a joystick. By twisting or tilting the joystick, the tensile wires undergo corresponding longitudinal displacements, the deflections of which serve to deform the tip of the guiding rod.

US published patent application 2013/0317300 A1 discloses a combination of a video laryngoscope and a video-supported, controllable guiding rod, onto which is pushed an intubation tube. The guiding rod projects beyond the intubation tube at the distal end, with a flexibly designed rod end section that is designed to be spatially deflectable, and encloses the viewing optics of the video-supported, controllable guiding rod.

US published patent application 2009/0050146 A1 discloses a manipulation unit for the positioning of an ETT by use of a guiding rod arranged inside the ETT, which guiding rod projects beyond the ETT at the distal end. In addition, the manipulation unit has a separation mechanism for purposes of separating the guiding rod from the ETT.

WO 2018/109033 A1 describes a device for inserting and positioning an ETT by use of an actively deformable guiding rod, the actively controllable element of which completely projects beyond the ETT at the distal end, and which can be individually deformed along two sections of the rod by use of separate cable pulls, in order to facilitate the tracheal insertion process.

Further intubation devices can be found in U.S. Pat. Nos. 9,888,832 B2 and 10,758,708 B2 and WO 2020/247386 A1, all of which have a controllable guiding rod. The distal section of the guiding rod is designed to move in a controlled manner, and, for technical reasons, must always project beyond the distal end of the ETT, as is the case for all the generic insertion devices described above. As determined by the system, the ETT has a larger outer diameter than the guiding rod. During the process of insertion of the ETT, the guiding rod is therefore first pushed forward through the vocal cord plane into the trachea, and the ETT is then pushed through the vocal cord plane, over the guiding rod that is already positioned in the trachea. Due to the difference in calibre between the outer diameters of the guiding rod and the ETT, the vocal cords are subject to a high risk of injury from the ETT, which is usually advanced blindly. Furthermore, the jumps in caliber and the different angles (pharynx-larynx-trachea) lead to mechanical obstacles, especially when advancing the ETT over the guiding rod on the larynx, causing the ETT to stick on the components of the larynx or vocal cords in the course of advancement and cannot be further advanced without further manipulation (retraction, rotation, renewed laryngoscopy, or the application of considerably higher thrust forces). This requires valuable time, which, if wasted unnecessarily, can lead to at least one of hypoxia, aspiration, and significant injuries, such as vocal cord damage, or bleeds. These bleeds can completely inhibit the intubation procedure due to the considerably reduced visibility, which in turn can result in a tracheotomy, or death.

In addition, with the guiding rods of current known art that are in in use, which are positioned in the endotracheal tube, the guiding rod must either be removed by a second person during the intubation procedure, or the ETT must be advanced by a second person, or the laryngoscope must be removed so that the ETT can then be advanced blindly. This is due to the fact that the person intubating the patient holds the laryngoscope in their left hand, and the ETT to be inserted in their right hand. This means that both hands are occupied, and a third hand is always missing.

### SUMMARY OF THE INVENTION

The invention is based on providing a medical instrument for the intubation of a patient, with an ETT enclosing a hollow channel, together with a guiding rod that is arranged along the hollow channel, which can be plastically deformed, at least in an axial portion, and has a distal and a proximal rod end, to which distal end of the rod a bidirectionally deformable guiding element is attached, with a distal and with a proximal element end, the proximal element end of which is joined to the distal end of the rod and is operatively connected to a control, such that the manipulation of the medical instrument for carrying out the entire intubation procedure, that is to say, the insertion and positioning of the ETT, together with a separation of the guiding rod from the ETT and the removal of the same, can be carried out by the intubating person with one hand, such that a third hand is not required at any time, and consequently there is no need for a second person to assist in the intubation procedure.

To achieve the object underlying the invention, a medical instrument for intubating a patient (or animal), with the features of the preamble of the instrument is designed such that an operating unit is attached to the proximal rod end of the guiding rod, with an actuator connected to the control, and with a push-pull mechanism that is securely in operative connection with the ETT in a releasable manner, and by which the ETT can be moved along the guiding rod, and such that during intubation of the ETT, together with the guiding rod arranged in the hollow channel of the ETT with the bidirectionally deformable guiding element attached thereto, the distal end of the ETT finishes with the distal element end, or the proximal element end merely projects beyond the distal end of the ETT in the form of a dome, so that when the guiding element is deformed, the ETT is actively deformed along its distal end section.

With the medical instrument in accordance with the invention, it is possible, for the first time, for an intubating person, after appropriate manual pre-shaping of the guiding rod based on empirical experience, to navigate the guiding rod together with the ETT, that is to say, the guiding rod is arranged within the ETT, with only one hand within the airway region adjacent to the pharynx, through the vocal cords in a gentle and targeted manner, and to position it in a correspondingly correct manner, whereby the ETT can in this manner be actively deformed by way of the guiding rod. The guiding element, which can be bidirectionally deformed at the distal end of the rod, can be bidirectionally deformed or curved in a controlled manner with the aid of the actuator attached to the operating unit, preferably with the aid of a single digit, for example the thumb, such that the distal end of the element can be deflected either in one direction or in the corresponding opposite direction, depending on the patient-specific airway geometry. The choice of length of the ETT, the guiding rod, and the bidirectionally deformable guiding element attached to the distal end of the guiding rod, are matched to each other so that when the ETT is intubated or inserted into the patient's airway, together with the guiding rod arranged inside the ETT, and the bidirectionally deformable guiding element which is attached to it, the distal end of the ETT finishes with the distal end of the element, that is to say, is largely flush with it, in the sense that the proximal end of the element, which is preferably shaped like a dome, projects in the form of a dome beyond the otherwise open distal ETT opening, that is to say, the proximal end of the element, in the form of a dome, projects minimally axially out of the ETT opening in a distal direction. In this manner, there is effectively no difference in caliber between the distal end of the guiding rod and the ETT, since they both pass through the vocal cord plane as a unit.

In addition, the medical instrument designed in accordance with the invention offers the intubating person the possibility of positioning the ETT into the trachea via the guiding rod and then separating it from the guiding rod, without the need for manual manipulation or assistance from another person. In a similar manner to the actuator operation, a push-pull mechanism is provided on the operating unit for the purpose of distal advancement of the ETT by way of the guiding rod into the trachea which can also be operated with the aid of just one finger, preferably with the aid of the index finger, in a similar manner to the actuator operation described above.

The advantageous one-handed operation of the medical instrument of the invention is particularly suitable for emergency medical interventions, where a second person is absent, or otherwise occupied. The preferred ergonomic design of the operating unit, with the manually operated actuator and the push-pull mechanism attached to it, creates the instrumental prerequisites for fast and gentle intubation, whereby interruption or termination of intubation procedures due to at least one of aspiration, and reduced oxygen supply, can be ruled out, or at least significantly reduced.

The “one-handed” operating concept of the medical instrument of the invention for intubation also opens up the possibility of using robots, which is of great interest, especially in times of pandemic, for example those caused by corona viruses such as (SARS-CoV-2, SARS, MERS), or influenza, or Ebola, in order to protect the intubating persons. For this purpose, the operating unit must be designed in a suitable manner in the form of a mechanical or mechatronic connecting flange, for the purpose of adaptation to, and actuation by, a robotic unit.

In a preferred form of embodiment of the medical instrument, the control connected on the one hand to the actuator, and on the other hand to the distal element end of the bidirectionally deformable guiding element, has at least two linear elements that transmit tensile forces, preferably in the form of cables or tapes, and which are operatively connected to the actuator such that, when the actuator is actuated, both linear elements can be deflected kinematically in opposite directions to one another. For this purpose, the actuator preferably has a rotary element mounted so as to rotate about an axis of rotation, on which the end sections of both linear elements are operatively connected to the rotary element in half-planes located opposite the axis of rotation. The distal end sections of the two linear elements transmitting tensile forces are each attached to the distal element end of the guiding element, which is preferably made of flexurally elastic material, at two attachment points located opposite the longitudinal axis of the flexurally elastic guiding element.

Both linear elements are preferably of the same length and in a neutral position, in which the guiding element, which is preferably of an elongated, flexurally elastic design, assumes a straight, that is to say, curvature-free, shape, that is to say, in a state largely taut and free of tensile forces. When the actuator is actuated, that is to say, when the rotary element rotates, one of the two linear elements is deflected in the proximal direction through a distance, creating a tensile force acting on the guiding element, while the opposite linear element is guided in the distal direction. As a result, the flexible guiding element bends in the direction of the linear element subjected to tensile force. In the same manner, the guiding element bends in exactly the opposite direction when the actuator is actuated, and the rotary element rotates in the opposite direction about the axis of rotation.

In another embodiment, the end sections of both linear elements that are in operative connection with the rotary element are connected to each other in one piece, that is, monolithically. In this case, the rotary element is designed in the form of a rotary disk with a groove-shaped circumferential edge, along the half-circumferential edge of which the cable or tape-shaped control runs in close contact.

As an alternative to the design of the control in the form of a mechanical Bowden cable solution, which interacts with the flexible guiding element as described above, it is also possible to bend or deform a suitably assembled, flexible guiding element in a controlled manner by electrical, pneumatic, or hydraulic, control signals. For this purpose, the bidirectionally deformable guiding element has a structural unit that is able to initiate a change in shape of the guiding element by the electrical, pneumatic, or hydraulic, control signals.

In one embodiment, the structural unit has at least one of the following electrical or mechatronic components that can be activated by electrical control signals: an electromotive transmission unit, an actuator-sensor unit, a piezoelectric material, a magnetostrictive material, a shape memory alloy, etc. For example, in the case of the use of a piezoelectric material, which is provided as an integral component on at least one of, along, and within, the guiding element, electrically initiated material contractions lead to changes in the shape of the guiding element. The electrical control signals can be applied to the piezoelectric material by an electrical circuit containing at least an electrical energy source and a switch unit. The electrical energy source and the switch unit serving as an actuator are preferably housed in the operating unit. Only two electrical cable paths run along the guiding rod in order to control the electrical component integrated in the guiding element for the purpose of changing its shape.

In the same manner, in alternative embodiments, pneumatically or hydraulically activatable components integrated within the guiding element, that is by at least one inflating or filling body or volume, can be activated by fluid lines running along the guiding rod which can be activated by fluid lines running along the guiding rod. In these cases, the pneumatic or hydraulic circuit, which the fluid circuit is suitably assembled for this purpose, contains a pump and a control valve serving as an actuator, which are part of the operating unit.

In a preferred form of embodiment, in order to make manipulation of the medical instrument as comfortable and fatigue-free as possible, the operating unit, which is designed as a handle, is constructed in the form of an ergonomically-shaped gripping body, which can be surrounded on one side by the thumb, and on the opposite side by the rest of the hand. To operate the actuator, this provides at least one first operating element which is ergonomically arranged and designed on the gripping body such that the first operating element can be operated by means of the thumb of a hand enclosing the gripping body. For this purpose, the first operating element is preferably designed in the form of a lever or adjustment wheel, which can be pivoted or adjusted by the thumb in either one direction or in the opposite direction.

In addition, a second operating element is ergonomically arranged and designed on the ergonomically designed handle such that the second operating element can be actuated by the index or middle finger of the hand enclosing the handle. The second operating element is used to actuate the push-pull mechanism, by means of which the ETT can be moved along the guiding rod. The second operating element is preferably designed in the form of a lever attached to the handle, which can be pulled in the direction of the handle by the index or middle finger, or pushed away from the handle in the opposite direction. The second operating element is preferably designed in the form of a ring, through which the index or middle finger can be inserted.

The second operating element designed in the above manner is kinematically connected to a push-pull rod via a transmission unit arranged in the handle, which is detachably fixed to the proximal section of the ETT. When the second operating element is deflected in the proximal direction in the direction of the handle, the transmission unit is able to deflect or push the push-pull rod and the ETT, which is detachably fixed to it, in the distal direction, and when the second operating element is deflected in the opposite direction, it is able to deflect or push the push-pull rod and the ETT, which is detachably fixed to it, in the proximal direction. The transmission unit preferably provides a gear wheel with external teeth, which is rotatably mounted about an axis of rotation, and in which both a rack connected to the second operating element, and the push-pull rod designed in the manner of a rack, engage. A preferred form of embodiment of the design of the transmission unit is illustrated and described in detail below with reference to the figures.

The guiding rod, which can be plastically and flexibly deformed in certain sections, has a length that corresponds approximately to the length of the ETT. By virtue of the inherent material flexibility of the ETT about its longitudinal axis, it is advantageous, but not necessary, for the flexurally elastic guiding element attached to the proximal end of the guiding rod to protrude at most minimally over the ETT at the distal end.

In a further embodiment, an observation medium is guided along the lumen through the guiding rod, through which the linear elements of the control means are guided, or a further lumen through the guiding rod, which can be an illumination catheter, a camera catheter, or a LIDAR sensor-supported catheter, for example. In this manner, it is possible to perform the intubation procedure while it is visually monitored, and to individually adapt the controlled curvature of the guiding element in situ to the spatial airway conditions in order to position the ETT gently and as quickly as possible.

### BRIEF DESCRIPTION OF THE DRAWINGS

Without limiting the general concept of the invention, the invention is described below by way of examples of embodiment with reference to the drawings.

FIG. 1 shows an example of a medical instrument in accordance with the invention for the intubation of a patient;

FIG. 2 shows a guiding rod with a bidirectionally deformable guiding element;

FIG. 3 shows a detailed view of the deformable guiding element; and

FIG. 4 shows a guiding rod with a bidirectionally deformable guiding element, with an electrically, pneumatically, or hydraulically, activatable unit.

### DETAILED DESCRIPTION OF THE INVENTION

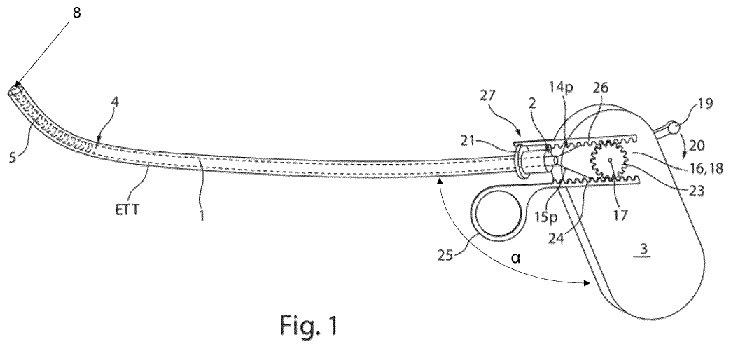

FIG. 1 illustrates a schematic overall view of the medical instrument for the intubation of a patient with ETT, which has a guiding rod 1, the proximal end 2 of which is connected to an operating unit 3 designed as a handle, and on the distal end 4 of which is arranged a bidirectionally deformable guiding element 5.

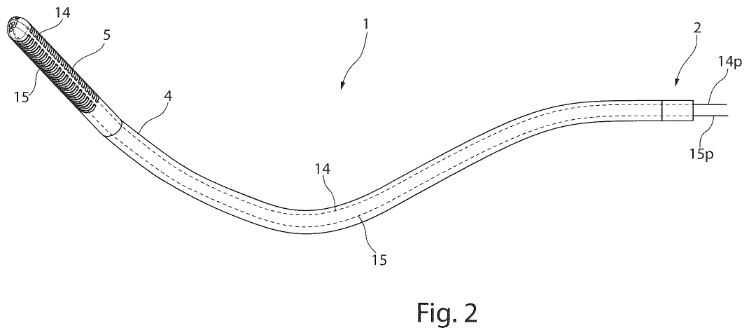

FIG. 2 shows a detailed view of the guiding rod 1 and the bidirectionally deformable guiding element 5 arranged at the distal end of the rod 4.

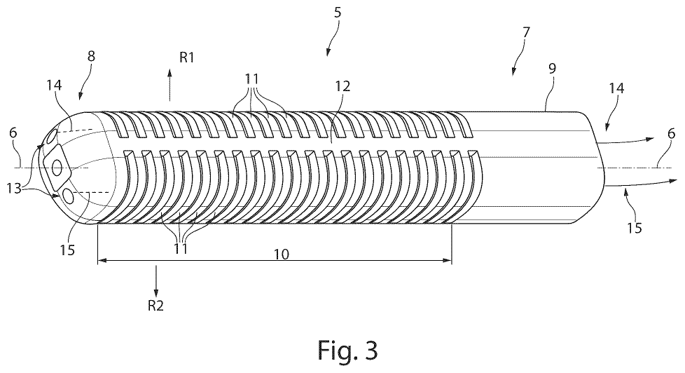

FIG. 3 shows a detailed view of a preferred form of embodiment of a bidirectionally deformable guiding element 5. Other statements make common reference to FIGS. 1 to 3.

The guiding rod 1, which is designed as a hollow channel, is preferably made of a plastically deformable material, so that an intubating person can initially curve the guiding rod 1 manually to achieve the gentlest possible oral insertion into the pharyngeal region and the adjoining airway, as shown in FIG. 2. As an alternative to designing the guiding rod 1 as a hollow channel, it is possible to design the guiding rod 1 in a cross-chamber construction, that is, the guiding rod 1 has a cross-shaped cross-section and is directly surrounded by the ETT. The cross-chambers running longitudinally within the ETT allow the tensile wires to run with relatively little resistance, while the cross-shaped cross-section of the guiding rod allows the tensile wires to be guided and provides the necessary physical rigidity.

The bidirectionally deformable guiding element 5, joined at the distal end of the rod 4, has a monolithically manufactured sleeve-like hollow body, which has a longitudinal axis 6, a proximal element end 7, and a distal element end 8, as shown in detail in FIG. 3. The proximal element end 7 is securely joined on one side to the distal rod end 4 of the guiding rod 1. The distal element end 8 is designed as a dome and has a largely closed surface in order to ensure that the guiding rod 1 is introduced into the airway region as smoothly as possible, and without any lesions, that is to say, to ensure an easy insertion of the guiding rod into the ETT. The axial length of the ETT is dimensioned so that during insertion of the ETT into the airway of a patient by use of the medical instrument, the ETT completely surrounds the guiding rod 1 and the guiding element 5, which is attached to it, in a bidirectionally deformable manner, along its axial extent. Advantageously, only the dome formed distal end 8 of the guiding element 5 projects axially through and beyond the distal end of the ETT, in order to make the intracorporeal advancement movement through the vocal cords and trachea as gentle as possible with the dome form, as can also be seen from FIG. 1.

The ETT is made of a material with inherent flexibility. The ETT can be bent about its longitudinal axis, and is thus able to follow a spatial shape determined by an initial plastic bending of the guiding rod 1, and a controlled deflection of the guiding element 5. One advantage of the medical instrument stems from the active spatial deformability of the distal end of the ETT, which is initiated by the guiding element 5, and enables a direct intracorporeal advancement of the ETT along the airway. Here the majority of the ETT can be preformed by use of the plastically deformable part of the guiding rod, in accordance with the patient-specific profile of the airway, whereby the distal element end 8 of the ETT remains movable for navigation purposes. This leads to a considerably faster procedure from the start of intubation up to the point at which the patient can be ventilated. In this manner, serious injury to the patient caused by a possible lack of oxygen can be avoided.

A further advantage ensues from the axial positional coincidence of the distal ends of the ETT and the bidirectionally deformable guiding element 5, as a result of which a caliber jump that irritates the vocal cords, which is unavoidable with generic intubation devices of known art, with a guiding rod 5 leading ahead from the ETT, does not occur. Thus, as required by the system, the diameter of the guiding rod is always smaller than the ETT finally to be positioned. In practice, this means that if the guiding rod precedes the ETT, it can be positioned in the trachea without any problems in most cases, but the subsequent ETT can often not be pushed past the vocal folds or vocal cords, due to the unsteady larger outer diameter, or can only be pushed past the vocal cords with force or rotational movements, that is to say, injury can result to the vocal cords. This can lead to hoarseness, possibly for the rest of the patient's life. Furthermore, surrounding tissue can be injured by the increased force exerted, especially in the case of emergency patients. This often results in acute bleeds, which can make vision and therefore intubation impossible. The consequence would be a tracheotomy, or, if this is unsuccessful, the death of the patient.

By virtue of the coordinated lengths of the ETT, guiding rod and guiding element, the distal end of the ETT finishes with the distal end of the guiding element, or the proximal end of the element just overlaps the distal end of the ETT in the form of a dome, and the ETT can be positioned directly through the vocal folds with its cross-sectional dimensions adapted to the patient, as is the case in an intubation without a guiding rod. In this manner, an operator can guide the ETT through the vocal cords in a way that is gentle on the airway, and can position the distal end of the ETT quickly and safely behind the vocal cords in the direction of intubation.

The guiding element 5 illustrated in FIG. 3 is a hollow body that is symmetrical about the longitudinal axis 6. In the region of the proximal element end 7, the guiding element 5 has an uninterrupted sleeve wall 9 for purposes of joining to the guiding rod 1 with the greatest possible dimensional stability; the sleeve wall 9 is adjoined in the distal direction by a bidirectionally deformable section 10. In the section 10, the sleeve wall is structured and has wall sections 11 that are formed like ribbed arches, and are axially separated from each other by distances orthogonal to the longitudinal axis 6, and are each connected on both sides to a sleeve wall web 12, which extends from the proximal side of the uninterrupted sleeve wall 9 to the distal element end 8. For completeness, it should be mentioned that opposite the sleeve wall web 12 visible in FIG. 3, a corresponding sleeve wall web is arranged on the rear side of the guiding element 5, which web is not visible in FIG. 3.

The bidirectionally deformable section 10 structured in the above manner and shown in FIG. 3 enables the guiding element 5 to be curved in the two oppositely oriented directions R1, R2, as shown by arrows, which in each case are oriented transversely to the longitudinal axis 6.

For the controlled deflection of the bidirectionally deformable guiding element 5 along one of the two directions of curvature R1, R2, an attachment structure 13 is attached to the distal element end 8 for purposes of fixing the respective distal element ends of the two tensile force-transmitting linear elements 14, 15. The linear elements 14, 15 are designed in the form of a cable or tape and run inside the guiding element 5, which is designed as a hollow body, in the proximal direction through the guiding rod 1, which is designed as a hollow channel, and adjoin the guiding element 5 in the proximal direction, see also FIG. 2.

The proximal end sections 14p, 15p of the linear elements are seen in 14, 15 and FIG. 1, protrude in the proximal direction from the guiding rod 1 and are connected to an actuator 16 integrated in the operating unit 3, which is designed as a handle. The actuator 16 illustrated in FIG. 1 has a rotary element 18 mounted such that it can rotate about an axis of rotation 17 which articulates an operating lever 19 that can be operated by the thumb of a hand enclosing the handle 3. When the operating lever 19 is pivoted in the pivoting direction 20 as shown in FIG. 1, the upper linear element 14 is pulled in the proximal direction, whereas the lower linear element 15 is guided in the distal direction. In this case, a tensile force acts along the upper linear element 14, which is able to bend the guiding element 5 attached to the distal side of the guiding rod 1 in the manner shown in FIG. 1. When the operating lever 19 is deflected in the opposite direction to the pivoting direction 20, the guiding element 5 reverses its curvature accordingly.

In FIG. 1, an endotracheal tube, ETT for short, is slid over the guiding rod 1. A ring-shaped connector 21, which is part of the ETT, is attached to the proximal end of the ETT. The connector 21 is in operative connection with a push-pull mechanism 22, also integrated in the handle 3, which is able to deflect the ETT along the guiding rod 1 manual actuation. The push-pull mechanism 22 has a toothed wheel 23 with external teeth, which is mounted to rotate about the axis of rotation 17, and which engages with a toothed rack 24, which is designed on one side as a ring-shaped second operating element 25, with which the index finger of a hand gripping the handle 3 can engage. On the other hand, opposite the toothed rack 24 a further toothed rack 26 engages with the external teeth of the toothed wheel 23; this is securely in operative connection in a releasable manner at the end with the collar 21 of the ETT by way of a detent mechanism 27.

By a deflection in the proximal direction of the toothed rack 24, the toothed rack 26, and the ETT connected to it, are pushed in the distal direction to the same extent. At the same time, it is possible to move the ETT in the proximal direction along the guiding rod 1 by a distal deflection of the toothed rack 24 by means of the second operating element 25.

Thus it is possible to insert the medical instrument with a preformed guiding rod 1 into a person's airway with just one hand and, by an appropriate actuation of the first control lever 19, preferably with the thumb, to bend the bidirectionally deformable guiding element 5 individually for the purpose of gentle insertion and easier navigation of the instrument into the patient, whereby the ETT is also bent along its extent surrounding the guiding element 5, and can thus be positioned intracorporeally in synchronization with the guiding rod 5. After appropriate positioning, the ETT can then be advanced in the distal direction into the trachea by using the second operating element 25, which can be actuated in a suitable manner with the index finger. Once the ETT has been positioned and fixed in position, for example in a manner known per se, with an inflatable balloon (a so-called cuff, not shown) attached to the ETT, the detent mechanism 27 is released from the collar 21 securely connected to the ETT and the guiding rod 1 is pulled in the proximal direction out of the ETT.

In particular, the shape and attachment of the handle 3 in the form of a “pistol” grip relative to the ETT that is to be handled with one hand, and the bidirectionally deformable guiding element 5 attached to the guiding rod 1, also contribute to ergonomically favourable and force-efficient manipulation for the intubation procedure. This makes it possible to deform the ETT tip bidirectionally during the entire intubation procedure, and to hold it in a specific shape. This is achieved by orienting the hand enclosing the handle 3, and thus the longitudinal extent that can be assigned to the handle 3, largely orthogonally to the direction of advance along the ETT. On the one hand, this ensures a more efficient power transmission and, on the other hand, larger deflection angles of up to 120° about the longitudinal axis of the ETT can be achieved. Typically, the longitudinal extent of the handle 3 is inclined relative to the longitudinal extent of the ETT by an angle a, where 90°≤α≤135° Due to this near-orthogonal alignment, the push-pull mechanism 22 sits approximately at the height of the detent mechanism 27, which establishes the connection to the ETT, resulting in an immediacy in the manipulation of the operating element 25 and the resulting ETT movement, with an associated precise manipulation of the ETT.

Since the guiding rod 5 ends in the proximal direction at approximately the same height as the ETT, and the handle 3 connects directly to the proximal end of the ETT, the medical device can have a compact overall length. As a result, lateral movements, for example, are transmitted very directly to the ETT, and only minimal losses of torsional or shear forces occur. The direct transmission of force gives the operator a precise haptic control, that is to say, the procedure for the insertion of the breathing tube can be carried out more quickly and safely, and the forces acting on the ETT are fed back more precisely, making vocal cord damage or even a puncturing of the trachea, less likely, for example. The compact design of the medical device in accordance with the solution makes it suitable for the first time for infant intubations, for example, as very fine haptic control is essential in these cases.

The design and attachment of the handle 3 allows six degrees of freedom to be achieved on the distal ETT tip with one hand, that is to say, bidirectional active bending by the first operating lever 19, right/left rotation by rotation of the entire handle, and axial forward and backward movement of the entire ETT by the second operating element 25. This allows the optimum angle for the ETT tip to be set for passage past the vocal cords, and, after passage past the vocal cords, to be moved further in the anatomically predetermined direction, in order to bend the ETT behind the vocal cords in the axial direction of the trachea.

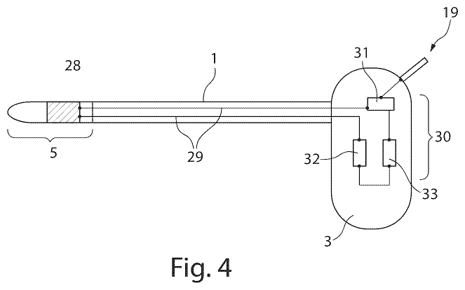

FIG. 4 illustrates a guiding rod 1 with an operating unit 3 attached at the proximal end to it, which is designed as a handle. An elastically flexible guiding element 5 is provided at the distal end of the guiding rod 1; this has a structural unit 28 as an integral component, which is capable of initiating a change in shape of the guiding element 5 by means of electrical, pneumatic, or hydraulic, control signals. For further explanation, it is assumed that the structural unit 28 can be operated and activated electrically, and is designed in the form of one of the following components: an electromotive transmission unit, an actuator-sensor unit, a piezoelectric material, a magnetostrictive material, a shape memory alloy, etc.

The unit 28 is connected to an electrical circuit 30 arranged in the operating unit 3 via two electrical cable paths 29, which are arranged along the guiding rod 1. In the case shown, the electrical circuit 30 contains an electrical energy source 31, a switch unit 32, which serves as an actuator, and can be actuated by the first operating lever 19, and an electrical control and monitoring unit 33.

Instead of the operating unit 3 illustrated in FIG. 4, which is designed as a handle, it is also conceivable to design the operating unit as a connecting flange for adaptation to, and actuation by, a robot unit. In this case, the control unit 28 integrated in the guiding element 5 is actuated by a control system provided on the robot, which also controls the robot motor system in order to create the conditions for autonomous intubation. Needless to say, this presupposes that a suitable sensor system is attached to the distal region of the guiding element. For example, as already mentioned above, it makes sense to guide an observation device through the lumen of the guiding rod, or another lumen through the guiding rod, for example in the form of an illumination catheter, a camera catheter and at least one of a catheter supported by a LIDAR sensor.

The second operating unit 25 and the push-pull mechanism 22 on the operating unit 3, which is designed as a handle, are not shown in FIG. 4 in the interests of clarity.

Overall, it should be noted that the rapid and correct positioning of the ETT plays an eminently vital role in the induction of anaesthesia, and emergency care and intensive care medicine, where problems can very quickly lead to life-threatening complications. In the event of a known or unknown difficult airway, a wide variety of methods and aids are used. In particular, there is a need both to bring the ETT into a specific anatomical shape using an internal passive guiding rod, and actively to control the ETT tip in order to pass through the vocal cord plane in a manner that is as free of injury as possible, and then position it in the axial direction of the trachea. The medical device in accordance with the solution makes the above measures possible for the first time without the need for assistance from a second person.

### LIST OF REFERENCE SYMBOLS

1 Guiding rod
2 Proximal rod end
3 Operating unit, handle
4 Distal rod end
5 Bidirectionally deformable guiding element
6 Longitudinal axis
7 Proximal element end
8 Distal element end
9 Interruption-free sleeve wall
10 Bidirectionally deformable section
11 Rib arch-like wall sections
12 Sleeve wall web
13 Attachment structure
14,15 Linear element
14p, 15p End of linear element
16 Actuator
17 Axis of rotation
18 Rotary element
19 First operating lever
20 Pivoting direction
21 Connector
22 Push-pull mechanism
23 Toothed wheel
24 Toothed rack
25 Second operating unit
26 Toothed rack
27 Detent mechanism
28 Unit
29 Electrical cable routes
30 Electrical circuit
31 Electrical energy source
32 Switch unit
33 Control and monitoring unit
R1, R2 Curvature directions
α Angle of inclination of the longitudinal direction of the operating unit relative to the ETT longitudinal extent

1-16. (canceled)

17. A medical instrument for the intubation of a patient, comprising:
an endotracheal tube with a distal end enclosing a hollow channel, a guiding rod disposed along the hollow channel which is plastically deformable at least in an axial portion and has distal and proximal rod ends, a bidirectionally deformable guiding element attached to the distal rod end which has a distal element end and a proximal element end, the proximal element end is joined to the distal rod end and is operatively connected to a control;
an operating unit attached to the proximal rod end, an actuator connected to the control, including a push-pull mechanism, operatively connected in a releasable manner to the endotracheal tube for moving the endotracheal tube along the guiding rod; and
the endotracheal tube and the guiding rod are disposed in the hollow channel of the endotracheal tube, the bidirectionally deformable guiding element being attached to the guiding rod, a distal end of the endotracheal tube ending with either the distal element end of the guiding element or the proximal element end of the guiding element projects beyond the distal end which is configured as a dome.

18. The medical instrument in accordance with claim 17, wherein:
the control includes at least two linear elements which transmit tensile forces, each linear element having an end fixed to the distal element end of the bidirectionally deformable guiding element, together with a linear end section located opposite the linear element end fixed to the guiding element, wherein the end section is moveable into an operative connection with an actuator, so the actuator when actuated causes both linear elements to be kinematically deflectable in opposite directions to one another.

19. The medical instrument in accordance with claim 18, wherein:
the actuator includes a rotary element which is rotatable about an axis of rotation, on which the linear end sections of each linear element operatively are connected with the rotary element in half-planes located opposite the axis of rotation.

20. The medical instrument in accordance with claim 18, wherein:
the two linear elements transmit tensile forces and are connected together in one piece at the linear end sections.

21. The medical instrument in accordance with claim 17, wherein:
the linear elements transmitting tensile forces are connected together in one piece at the linear end sections.

22. The medical instrument in accordance with claim 17, wherein:
the operating unit includes a handle or a connecting flange adapted for connection to a robot.

23. The medical instrument in accordance with claim 18, wherein:
the operating unit includes a handle or a connecting flange adapted for connection to a robot.

24. The medical instrument in accordance with claim 19, wherein:
the operating unit includes a handle or a connecting flange adapted for connection to a robot.

25. The medical instrument in accordance with claim 20, wherein:
the operating unit includes a handle or a connecting flange adapted for connection to a robot.

26. The medical instrument in accordance with claim 22, wherein:
the handle comprises an ergonomically shaped gripping body;
the actuator provides at least a first operating element, ergonomically arranged on the gripping body so that the first operating element is actuatable by a thumb of a hand enclosing the gripping body; and
the push-pull mechanism comprises at least one second operating element which ergonomically arranged on the gripping body so that the at least one second operating element is actuatable by an index or middle finger of a hand enclosing the gripping body.

27. The medical instrument in accordance with claim 26, wherein:
the second operating element includes a lever attached to the gripping body which is kinematically connected via a transmission on the gripping body to a push-pull rod which is releasably fixed to a proximal section of the endotracheal tube; and
the transmission unit pushes the push-pull rod, and the endotracheal tube is detachable in a distal direction when the lever is deflected proximally in a direction of the gripping body, and when the lever is deflected in an opposite direction, the push-pull rod and the endotracheal tube which are detachably connected thereto are pushed in a proximal direction.

28. The medical instrument in accordance with claim 26, wherein:
the transmission comprises a gearwheel with external teeth, mounted to rotate about an axis of rotation, and a toothed rack connected to the lever with the push-pull rod engaging the toothed rack.

29. The medical instrument in accordance with claim 26, wherein:
the bidirectionally deformable guiding element includes at least in a region of a flexible elastic material, has a longitudinal extension from the proximal to the distal end with a longitudinal axis of the guiding element, and provides an attachment structure at the distal element end with an extension oriented symmetrically orthogonal to the longitudinal axis; and
the distal element ends of the two tensile force-transmitting linear elements are articulated at attachment points spaced at right angles to the longitudinal axis.

30. The medical instrument in accordance with claim 17, wherein:
the bidirectionally deformable guiding element initiates a change in shape of the guiding element by one of an electrical, pneumatic, or hydraulic, control signals;
the control has a circuit connected to the operating unit; and
the actuator attached to the operating unit includes part of the circuit.

31. The medical instrument in accordance with claim 30, wherein:
the guiding element comprises at least one of a mechatronic unit, an electromotive transmission, an actuator-sensor, a piezoelectric material, a magnetostrictive material, a shape memory alloy, an inflatable body, a fluid line.

32. The medical instrument in accordance with claim 17, wherein:
the guiding rod is penetrated by a lumen, through which the control runs.

33. The medical instrument in accordance with claim 32, wherein:
the guiding rod and a lumen extending along the guiding element are guided by an observation medium along the lumen or a further lumen.

34. The medical instrument in accordance with claim 33, wherein:
the observation medium includes at least one of an illumination catheter, a camera catheter, a LIDAR catheter, and a sensor catheter.

35. The medical instrument in accordance with claim 22, wherein:
the handle has a longitudinal extension including an angle a relative to a longitudinal extension of the endotracheal tube, where 90°≤α≤135°.

36. The medical instrument in accordance with claim 17, wherein:
the push-pull mechanism and the proximal region of the endotracheal tube partially overlap on the longitudinal extent of the endotracheal tube.
